(19) United States
(12) Patent Application Publication
(10) Pub. No.: US 2023/0052453 A1
(43) Pub. Date: Feb. 16, 2023

(54) COMPOSITIONS AND METHODS FOR RELIEVING EFFECTS OF ALCOHOL CONSUMPTION

(71) Applicant: MOXY DISTRIBUTION, INC., Newport Beach, CA (US)

(72) Inventor: Amber Adrianne Danz, Irvine, CA (US)

(21) Appl. No.: 17/881,527

(22) Filed: Aug. 4, 2022

Related U.S. Application Data
(60) Provisional application No. 63/203,973, filed on Aug. 5, 2021.

(51) Int. Cl. A61K 31/352; A61K 9/00; A61K 31/44; A61K 31/706; A61K 31/05; A61K 47/12; A61K 47/14; A61K 47/24; A61P 25/32

(52) U.S. Cl. CPC A61K 31/352; A61K 9/0053; A61K 31/44; A61K 31/706; A61K 31/05; A61K 47/12; A61K 47/14; A61K 47/24; A61P 25/32

(57) ABSTRACT

Compositions and methods for preventing or relieving hangover symptoms are provided. The composition comprises a flavonoid such as dihydromyricetin, an antioxidant comprising a substituted pyridine, such as emoxypine succinate, and a mononucleotide such as nicotinamide mononucleotide. The composition may be administered to a subject in need thereof as an oral formulation.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND OF THE INVENTION

### Field of the Invention

The present invention relates to compositions and method for relieving effects of alcohol consumption such as hangover. More specifically, it relates to a hangover relieving formulation.

### Description of the Related Art

For many people, a night of drinking can be followed by an unpleasant morning with hangover symptoms. Typical hangover symptoms headache, fatigue, dehydration, insomnia, weakness, muscle aches, nausea, dizziness, vertigo, sensitivity to light and sound, and mental fog. The severity of the hangover can vary among people who consume alcohol. Many factors may contribute to the hangover symptoms. For example, alcohol can increase urination and promote excess lost of fluids, which causes mild dehydration. Alcohol can also irritate the lining of the stomach directly and increase inflammation in the body. When the alcohol is metabolized by the liver, a toxic compound acetaldehyde is created, and can contribute to inflammation in the liver, pancreas, brain, gastrointestinal track and possibly some other organs if it is left in the blood stream for too long. After alcohol is broken down into acetaldehyde, the acetaldehyde is metabolized into an acetate anion which is less toxic and is easily eliminated by the body. Another effect is that alcohol can cross the blood brain barrier and rapidly downregulates GABA_{A}Rs. This has been known to cause altered physiology and pharmacology consistent with symptoms of tolerance and withdrawal.

### SUMMARY OF THE INVENTION

Provided herein is an oral composition for preventing or relieving hangover symptoms comprising dihydromyricetin, emoxypine succinate, and nicotinamide mononucleotide.

In some embodiments, the dihydromyricetin is an extract of at least one botanical selected from the group consisting of Ampelopsis japonica, Ampelopsis megalophylla, Ampelopsis grossedentata, Cercidiphyllum japonicum, Hovenia dulcis, Rhododendron cinnabarinum, Salix sachalinensis, a Pinus species, and a Cedrus species.

In some embodiments, the dihydromyricertin in the oral composition may have a weight percentage of between about 10% and about 30%. In some embodiments, the emoxypine succinate in the oral composition may have a weight percentage of between about 2% and about 8%. In some embodiments, the nicotinamide mononucleotide in the oral composition may have a weight percentage of between about 2% and about 11%.

In some embodiments, the dihydromyricertin in the oral composition may have a weight percentage of between about 18% and about 25%, the emoxypine succinate in the oral composition may have a weight percentage of between about 3% and about 5%, and the nicotinamide mononucleotide in the oral composition may have a weight percentage of between about 4% and about 8%.

In some embodiments, the oral composition further comprises trimethylglycine. In some embodiments, the trimethylglycine in the oral composition may have a weight percentage of between about 2% and about 6%.

In some embodiments, the oral composition further comprises a resveratrol blend. In some embodiments, the resveratrol blend in the oral composition may have a weight percentage of between about 4% and about 9%.

In some embodiments, the oral composition may further comprise caprylic acid, medium chain triglycerides, and sunflower lecithin.

In some embodiments, the weight ratio of dihydromyricetin to emoxypine succinate in the oral composition is from about 8:1 to about 3:1. In some embodiments, the weight ratio of dihydromyricetin to nicotinamide mononucleotide in the oral composition is from about 5:1 to about 2:1. In some embodiments, the weight ratio of emoxypine succinate to nicotinamide mononucleotide in the oral composition is from about 1:1 to about 1:2.

Also provided herein is a method for relieving or preventing hangover symptoms comprising administering an oral composition disclosed herein to a subject in need thereof.

In some embodiments, each dose comprises about 825 mg to about 1800 mg of dihydromyricetin, about 150 mg to about 400 mg of emoxypine succinate, and about 275 mg to about 600 mg of nicotinamide mononucleotide.

In some embodiments, a first half dose is administered prior to alcohol consumption, and the second half dose is administered after the alcohol consumption. In some embodiments, the first half dose is administered immediately or up to about 1 hour prior to alcohol consumption. In some embodiments, the second half dose is administered immediately or up to 2 hour after alcohol consumption.

In some embodiments, one dose of the oral composition is administered to the subject before or after alcohol consumption. In some embodiments, less than one dose of the oral composition is administered to the subject.

### DETAILED DESCRIPTION OF THE EMBODIMENTS

Several embodiments of the present disclosure are related to compositions and methods for preventing or relieving hangover symptoms.

### Compositions

The composition for preventing or relieving hangover symptoms comprises a flavonoid such as dihydromyricetin, an antioxidant comprising a substituted pyridine, such as emoxypine succinate, and a mononucleotide such as nicotinamide mononucleotide.

Dihydromyricetin (DHM), is also known as ampelopsin and has the IUPAC name (2R,3R)-3,5,7-trihydroxy-2-(3,4,5-trihydroxyphenyl)-2,3-dihydrochromen-4-one. DHM is a compound belonging to the flavanonol class of compounds, which are one type of the flavonoids. DHM can be found in the Ampelopsis species japonica, megalophylla, and grossedentata; Cercidiphyllum japonicum, Hovenia dulcis; Rhododendron cinnabarinum; some Pinus species; some Cedrus species, and Salix sachalinensis.

In some embodiments, the DHM suitable for use herein is an extract of Hovenia dulcis. DHM and the Hovenia dulcis extract have been shown to ameliorates alcohol-induced liver injuries and promotes alcohol (EtOH) elimination by increasing the activities of alcohol dehydrogenase (ADH) and acetaldehyde dehydrogenase (ALDH) (Shen, Y. et al., J Neurosci. 2012 Jan. 4; 32(1): 390-401).

The structural features of DHM give the DHM molecule highly hydrophilic properties. Studies have also shown that DHM is poorly absorbed into the bloodstream, thus has a low bioavailability. The bioavailability after oral administration to rats was shown to be only 4.02%. In addition, the time required for DHM to reach peak plasma concentration is 2.67 hour after oral administration at a dose of 20 mg/kg. (Lu, L. et al., Pharm. Biol. 55, 657-662).

In some embodiments, DHM may have a weight percentage of between about 10% and about 30%, between about 15% and about 25%, between about 18% and about 25%, or about 20%, based on the total weight of the composition.

Emoxypine succinate is an organic salt and an antioxidant having the IUPAC name 2-ethyl-6-methyl-3-hydroxypyridine. Emoxypine succinate is also known as Mexidol and Mexifin. Its chemical structure resembles that of pyridoxine which is a type of vitamin B6. Emoxypine has beneficial physiological effects that include anti-atherosclerotic, anti-inflammatory, cardioprotective, improving cerebral blood circulation, inhibiting thrombocyte aggregation, and lowering cholesterol levels. Emoxypine has beneficial psychotropic effects that include anxiolytic, anti-stress, anticonvulsant, nootropic, and neuroprotective. Emoxypine inhibits free radical oxidation of biomembrane lipids and increases the activity of antioxidant enzymes.

In some embodiments, emoxypine can affect the amount of monoamines in the brain. In some embodiments, emoxypine increases the amount of dopamine in the brain. Regulation of monoamines and dopamine provides neuroprotection and induces mental clarity and well-being. In some embodiments, emoxypine modulates receptor complexes of the brain membranes by increasing the affinity of the receptors for neurotransmitters or other ligands. In some embodiments, the receptor complexes include benzodiazepine receptors, gamma aminobutyric acid (GABA) receptors, acetylcholine receptors, and any combination of the foregoing. In certain embodiments, the receptor complexes are GABA receptors. In some embodiments, emoxypine increases binding between DHM and receptor complexes of the brain membranes. In certain embodiments, emoxypine increases binding between DHM and GABA receptors. The increased binding of DHM and GABA reception may increase the bioavailability of DHM. In some embodiments, emoxypine increases the bioavailability of DHM. In some embodiments, administration of a combination of DHM and emoxypine produces a hangover relieving effect, an anti-intoxication effect, or both.

In some embodiments, emoxypine succinate may have a weight percentage of between about 2% and about 8%, between about 2% and about 6%, between about 3% and about 5%, or about 4.1%, based on the total weight of the composition.

Nicotinamide mononucleotide (NMN) is a derivative of niacin and is a nucleotide derived from ribose and nicotinamide. NMN is the direct precursor of the essential molecule nicotinamide adenine dinucleotide (NAD+) and NMN is converted into NAD+ when ingested into the body. NMN thus functions to increase NAD+ levels in the cells of the body. NAD+ is an essential component of a wide range of biochemical processes including metabolic processes, such as glycolysis, the TCA Cycle (Krebs cycle, citric acid cycle), and the electron transport chain. After consumption of an alcoholic beverage, ethanol can deplete NAD+ levels in the body. Certain enzymes in the liver require NAD+ as a cofactor in order to de-acetylate groups of acetylated proteins. The depleted NAD+ levels can impede proper liver function leading to injury of the liver and possible toxicity that presents as painful hangover symptoms. NAD+ is also essential for the dehydrogenation of the acetaldehyde, which is toxic and which causes hangover symptoms, as described elsewhere herein. NMN, when ingested orally, is metabolized by the strong acids present in the stomach and has very low bioavailability as an oral formulation. In some embodiments, when NMN is placed in an enteric capsule, it is more readily absorbed in the small intestine and can be effectively converted by the body into additional NAD+, so that more NAD+ is available to be used in the dehydrogenation of acetaldehyde.

In some embodiments, nicotinamide mononucleotide may have a weight percentage of between about 2% and about 11%, between about 3% and about 9%, between about 4% and about 8%, or about 5.5%, based on the total weight of the composition.

In some embodiments, the combination of dihydromyricetin, emoxypine succinate, and nicotinamide mononucleotide provides an unexpected result of increased efficacy or effectiveness of preventing the occurrence of hangover following alcohol consumption. In some embodiments, the combination of dihydromyricetin, emoxypine succinate, and nicotinamide mononucleotide provides an unexpected result of increased efficacy or effectiveness of relieving at least some of the hangover symptoms following alcohol consumption.

It has been clinically shown that dihydromyricetin has an effect on alcohol intoxication in rats. However, dihydromyricetin has poor bioavailability in humans. When combining dihydromyricetin with emoxypine succinate, the binding ability of dihydromyricetin to GABA receptor is enhanced, which increases the bioavailability of dihydromyricetin. When nicotinamide mononucleotide is combined with dihydromyricetin and emoxypine succinate, it contributes additional NAD+ to cells which functions as a building block for enzymes that metabolize alcohol and acetaldehyde. As a result, the combination further enhances the composition's effectiveness in preventing or reducing the hangover symptoms.

In some embodiments, the composition may further comprise trimethylglycine. Trimethylglycine may be useful for donating methyl groups to NMN, which can be depleted as it is converted to NAD+. In some embodiments, trimethylglycine may have a weight percentage of between about 2% and about 6%, between about 3% and about 5%, between about 4% and about 4.5%, or about 4.1%, based on the total weight of the composition.

In some embodiments, the composition may further comprise a resveratrol blend. The resveratrol blend consists of trans-resveratrol, quercetin, grape skin extract, green tea extract, red wine extract and acai fruit extract. In some embodiments, the resveratrol blend may serve to block enzyme CD38, which is an NAD+ catalase. In some embodiments, it is preferable that the level of NAD+ remain higher while alcohol is present in the bloodstream. This may boost its ability to reduce acetaldehyde into less toxic components. In some embodiments, while the precursor to NAD+, NMN, serves to increase the NAD+ levels, a resveratrol blend can be added to prevent the additional NAD+ levels from being broken down before use. This excess is allowed to be converted into additional ADH and ALDH. In some embodiments, the additional ADH and ALDH enzymes can metabolize toxic alcohol byproducts faster.

In some embodiments, resveratrol blend may have a weight percentage of between about 4% and about 9%, between about 4% and about 8%, between about 5% and about 7.5%, or about 6.8%, based on the total weight of the composition.

The composition may further include other excipients or inactive ingredients. For example, caprylic acid, medium chain triglycerides, sunflower lecithin, and a combination thereof may also be included. In some embodiments, the composition may further comprise caprylic acid. In some embodiments, caprylic acid may have a weight percentage of between about 2% to about 5%, between about 3% and about 5.5%, or between about 3.5% and about 5%, based on the total weight of the composition. In some embodiments, the composition may further comprise medium chain triglycerides. In some embodiments, medium chain triglycerides may have a weight percentage of between about 30% to about 50%, between about 35% to about 50%, or at about 48%. As used herein, a “medium chain triglyceride” contain fatty acids having a chain length of 6-12 carbon atoms. In some embodiments, the composition may further comprise sunflower lecithin. In some embodiments, sunflower lecithin may have a weight percentage of between about 6% to about 13%, between about 6% to about 10%, or at about 6.8%.

In some embodiments, the composition may have a weight ratio of dihydromyricetin to emoxypine succinate from about 8:1 to about 3:1, from about 6:1 to about 4:1, or about 5:1. In some embodiments, the composition may have a weight ratio of dihydromyricetin to nicotinamide mononucleotide is from about 5:1 to about 2:1, from about 4.5:1 to about 2.5:1, or about 3.75:1. In some embodiments, the composition may have a weight ratio of emoxypine succinate to nicotinamide mononucleotide is from about 1:1 to about 1:2, about 1:1.1 to about 1:1.8, or about 1:1.3.

In some embodiments, the composition disclosed herein is for oral administration. Thus in some embodiments, the composition is an oral composition. In some embodiments, the composition is encapsulated in an enteric capsule. In some embodiments, the enteric capsule may provide delayed release of the composition. In some embodiments, the enteric capsule may preserve the bioavailability and increase absorption. In some embodiments, the medium chain triglycerides (i.e., MCT oil) are quickly and easily absorbed. By encapsulating the composition in the enteric capsule, it may be more easily absorbed by the body.

In some embodiments, the oral composition may be in a powder form. The powder formulation may be dissolved or mixed in with water or other beverages, including alcoholic beverages. In some embodiments, the oral composition may be in a liquid form. In some embodiments, the oral composition may be a liquid suspension.

### Methods

Provided herein includes a method for relieving or preventing hangover symptoms. The method includes administering an oral composition as described herein to a subject in need thereof. For example, the method includes administering an oral composition comprising a flavonoid such as dihydromyricetin, an antioxidant comprising a substituted pyridine, such as emoxypine succinate, and a mononucleotide such as nicotinamide mononucleotide. The subject in need of the composition includes human subjects, and include those who anticipate consuming alcoholic beverages within the next few hours, or those who is about to consume alcoholic beverages. In some embodiments, the subject in need thereof may be a person who has started consuming alcoholic beverages. In some embodiments, the subject in need thereof may be a person who has finished consuming alcoholic beverages.

The full dose of the composition may be administered to the subject in need thereof at once or as divided dose. The composition may be administered to the subject in need thereof during the alcohol consumption, prior to the alcohol consumption, and/or after the alcohol consumption. For those who plan to do some light drinking, such as up to about 2 or about 3 drinks, less than the full dose may be enough to reduce or prevent hangover symptoms.

In some embodiments, the full dose may be divided up for administration prior to and after the consumption of alcoholic beverages. In some embodiments, the first half of the dose of the composition may be administered prior to the alcohol consumption, and the second half of the dose may be administered after the alcohol consumption. In some embodiments, the first half dose of the composition may be administered immediately prior to the alcohol consumption, or up to 1 hour prior to the alcohol consumption. For example, immediately, up to about 20 minutes, up to about 30 minutes, up to about 40 minutes, up to about 50 minutes, or up to about an hour prior to the alcohol consumption. In some embodiments, the first half dose of the composition may be administered between about 0 minute to about 60 minutes, about 10 minutes to about 50 minutes, about 20 minutes to about 40 minutes, or about 30 minutes prior to alcohol consumption. In some embodiments, the second half dose may be administered immediately after consuming the last alcoholic beverage, or up to 2 hours after the last alcoholic beverage. For example, immediately, up to about 10 minutes, up to about 20 minutes, up to about 30 minutes, up to about 40 minutes, up to about 50 minutes, up to about 60 minutes, up to about 70 minutes, up to about 80 minutes, up to about 90 minutes, up to about 100 minutes, up to about 110 minutes, or up to about 120 minutes after consuming the last alcoholic beverage.

In some embodiments, the full dose may be administered to the subject in need thereof at the same time, either prior to or after alcohol consumption. For example, in some embodiments, immediately, up to about 10 minutes, up to about 20 minutes, up to about 30 minutes, up to about 40 minutes, up to about 50 minutes, or up to about an hour prior to the alcohol consumption. In other embodiments, the full dose may be administered to the subject in need thereof immediately, or up to about 2 hours after the last alcoholic beverage consumption. For example, immediately, up to about 10 minutes, up to about 20 minutes, up to about 30 minutes, up to about 40 minutes, up to about 50 minutes, up to about 60 minutes, up to about 70 minutes, up to about 80 minutes, up to about 90 minutes, up to about 100 minutes, up to about 110 minutes, or up to about 120 minutes after the last alcoholic beverage.

In some embodiments, administering less than the full dose may be enough to prevent or reduce the hangover symptoms. For example, down to ⅙ of a dose, down to ⅓ of a dose, down to half of a dose, down to ⅔ of a dose, down to ⅚ of a dose may be administered. In some embodiments, any portion of one dose may be administered prior to consumption of alcohol and/or after the consumption of alcoholic beverages. The timing for the administration of less than the full dose is the same as described above for the full dose. In some embodiments, a portion of the dose may also be administered during the consumption of alcoholic beverage.

The effect of the composition disclosed herein may last for at least about 8 hours after the full dose of the composition is taken. In some embodiments, the effect of the composition may last for at least about 7 hours, at least about 6 hours, at least about 5 hours, at least about 4 hours, or at least about 3 hours.

### Dosage

The composition may be formulated into an oral dosage form such as a capsule or a tablet. A tablet or a capsule can also have an enteric coating to provide extended or delayed release. In some embodiments, a tablet or a capsule can have two or more layers, wherein at least one layer is enteric coating. A tablet or a capsule can be formulated to provide extended release. The composition may also be formulated into an oral dosage form such as a powder, which can be dissolved in or mixed into a beverage or water for administration. The composition may also be formulated into an oral dosage form such as a liquid.

In some embodiments, each dose of the composition may include about 825 mg to about 1800 mg, about 850 mg to about 1700 mg, about 875 mg, or about 1500 mg of dihydromyricetin. In some embodiments, each dose may also include about 150 mg to about 400 mg, about 160 mg to about 350 mg, or about 300 mg of emoxypine succinate. In some embodiments, each dose may also include about 275 mg to about 600 mg, about 300 mg to about 500 mg, or about 400 mg of nicotinamide mononucleotide.

In some embodiments, the dose may further include about 150 mg to about 350 mg, about 180 mg to about 300 mg, or about 300 mg of trimethylglycine. In some embodiments, the dose may further include about 300 mg to about 600 mg, about 400 mg to about 550 mg, or about 500 mg of resveratrol blend. In some embodiments, the dose may further include about 150 mg to about 350 mg, about 200 mg to about 325 mg, or about 300 mg of caprylic acid. In some embodiments, the dose may further include about 2400 mg to about 3500 mg, about 2600 mg to about 3500 mg, or about 2800 mg of medium chain triglycerides. In some embodiments, the dose may further include about 500 mg to about 900 mg, about 600 mg to about 800 mg, or about 500 mg to about 700 mg of sunflower lecithin.

The composition may be administered up to about 1 hour prior to consuming alcoholic beverages. In some embodiments, the composition may be administered immediately before or when begin to consume alcoholic beverages. In some embodiments, the composition may be administered from immediately after the drinking, up to about 2 hours of the drinking. In some embodiments, the composition may be administered during while drinking.

In some embodiments, the full dose is for moderate to heavy drinking, for example, in excess of about 3 drinks. In some embodiments, only half dose is needed for light drinking, such as consuming about 3 drinks or less.

In some embodiments, the packet is configured to include one dose of the composition. In some embodiments, two packets, each configured to contain half of the dose, may be package together for distribution for individual use. In some embodiments, multiple one-dose packets or half-dose packets may be packaged into a container, box, or bag for distribution. In some embodiments the packet is a foil packet. In some embodiments, the packet may have a dimension of about 3 inches by about 3 inches.

### EXAMPLES

### Example 1

An example of one dose of the composition has the following ingredients:

| Ingredients | Weight (mg) | Weight % |
|---|---|---|
| Dihydromyricetin | 1750 | 24.1% |
| Nicotinamide mononucleotide | 600 | 8.3% |
| Resveratrol blend | 500 | 6.9% |
| Emoxypine Succinate | 300 | 4.1% |
| Trimethylglycine | 300 | 4.1% |
| Caprylic Acid | 300 | 4.1% |
| Medium Chain Triglycerides; | 2800 | 38.6% |
| aka by CL as “MCT oil” | | |
| Sunflower lecithin | 700 | 9.7% |
| Total | 7000 | 100% |

### Example 2

Another example of one dose of the composition has the following ingredients:

| Ingredients | Weight (mg) | Weight % |
|---|---|---|
| Dihydromyricetin | 1500 | 20.5% |
| Nicotinamide mononucleotide | 400 | 5.5% |
| Resveratrol blend | 500 | 6.8% |
| Emoxypine Succinate | 300 | 4.1% |
| Trimethylglycine | 300 | 4.1% |
| Caprylic Acid | 300 | 4.1% |
| Medium Chain Triglycerides; | 3500 | 47.9% |
| aka by CL as “MCT oil” | | |
| Sunflower lecithin | 500 | 6.8% |
| Total | 7300 | 100% |

### Example 3

Initial studies of the effectiveness of the formulation containing DHM, emoxypine succinate and nicotinamide mononucleotide on reducing hangover symptoms were conducted. A group of participants were instructed to administer a composition containing one half dose which is 750 mg DHM 200 mg nicotinamide mononucleotide and 150 mg emoxypine succinate, and a control group of participants were instructed to administer a composition containing one half dose of 750 mg DHM 30 minutes prior to consuming 1 alcoholic beverage every 30 minutes, up to 3 hours, which was a total of 6 alcoholic drinks. The blood alcohol content (BAC) of the participants was measured once every 15 minutes after each drink. Participants were also administered a verbal processing test. It was found that individuals who were administered DHM and nicotinamide mononucleotide and emoxypine succinate sustained lower intoxication levels than individuals who were administered DHM alone.

The differential in blood alcohol content between the participants receiving DHM and nicotinamide mononucleotide and emoxypine succinate and the participants in control group were substantial as show in Table 1.

TABLE 1

| | | DHM, NMN, | | |
|---|---|---|---|---|
| | | EMOXYPINE | CONTROL | |
| | | SUCCINATE | DHM ALONE | |
| ELAPSED | CUMULATIVE | MEAN BAC | MEAN BAC | |
| TIME | DRINKS | LEVELS | LEVELS | DIFFERENTIAL |
| 00 h 15 m | 1 | 0.014% | 0.022% | 0.008% |
| 00 h 45 m | 2 | 0.033% | 0.051% | 0.018% |
| 01 h 15 m | 3 | 0.044% | 0.062% | 0.018% |
| 01 h 45 m | 4 | 0.064% | 0.087% | 0.023% |
| 02 h 15 m | 5 | 0.079% | 0.109% | 0.030% |
| 02 h 45 m | 6 | 0.092% | 0.131% | 0.039% |

In addition, participants who were administered a composition with DHM, emoxypine succinate and nicotinamide mononucleotide reported having more reduced symptoms of alcohol withdrawal the next day, if not eliminated, compared to those who were administered a composition with DHM alone. Symptoms measured included headache, fatigue, brain fog, poor mood, dizziness, sensitivity to light, and nausea. The administration of a composition with DHM, nicotinamide mononucleotide and emoxypine succinate resulted in significantly fewer symptoms of alcohol withdrawal compared to administered composition with DHM only.

What is claimed is:

1. An oral composition for preventing or relieving hangover symptoms comprising dihydromyricetin, emoxypine succinate, and nicotinamide mononucleotide.

2. The oral composition of claim 1, wherein the dihydromyricetin is an extract of at least one botanical selected from the group consisting of Ampelopsis japonica, Ampelopsis megalophylla, Ampelopsis grossedentata, Cercidiphyllum japonicum, Hovenia dulcis, Rhododendron cinnabarinum, Salix sachalinensis, a Pinus species, and a Cedrus species.

3. The oral composition of claim 1, wherein the dihydromyricetin is an extract of Ampelopsis grossedentata.

4. The oral composition of claim 1, wherein the dihydromyricertin has a weight percentage of between about 10% and about 30%.

5. The oral composition of claim 1, wherein the emoxypine succinate has a weight percentage of between about 2% and about 8%.

6. The oral composition of claim 1, wherein the nicotinamide mononucleotide has a weight percentage of between about 2% and about 11%.

7. The oral composition of claim 1, wherein the dihydromyricertin has a weight percentage of between about 18% and about 25%, the emoxypine succinate has a weight percentage of between about 3% and about 5%, and the nicotinamide mononucleotide has a weight percentage of between about 4% and about 8%.

8. The oral composition of claim 1, further comprising trimethylglycine.

9. The oral composition of claim 8, wherein the trimethylglycine has a weight percentage of between about 2% and about 6%.

10. The oral composition of claim 1, further comprising a resveratrol blend.

11. The oral composition of claim 10, wherein the resveratrol blend has a weight percentage of between about 4% and about 9%.

12. The oral composition of claim 1, further comprising caprylic acid, medium chain triglycerides, and sunflower lecithin.

13. The oral composition of claim 1, wherein the weight ratio of dihydromyricetin to emoxypine succinate is from about 8:1 to about 3:1.

14. The oral composition of claim 1, wherein the weight ratio of dihydromyricetin to nicotinamide mononucleotide is from about 5:1 to about 2:1.

15. The oral composition of claim 1, wherein the weight ratio of emoxypine succinate to nicotinamide mononucleotide is from about 1:1 to about 1:2.

16. The oral composition of claim 1, wherein the oral composition is formulated as an enteric capsule.

17. A method for relieving or preventing hangover symptoms comprising administering an oral composition of claim 1 to a subject in need thereof.

18. The method of claim 17, wherein less than one dose of the oral composition is administered to the subject.

19. The method of claim 18, wherein each dose comprises about 825 mg to about 1800 mg of dihydromyricetin, about 150 mg to about 400 mg of emoxypine succinate, and about 275 mg to about 600 mg of nicotinamide mononucleotide.

20. The method of claim 19, wherein each dose is formulated into six enteric capsules.

21. The method of claim 18, wherein a first half dose is administered prior to alcohol consumption, and the second half dose is administered after the alcohol consumption.

22. The method of claim 21, wherein the first half dose is administered immediately or up to about 1 hour prior to alcohol consumption.

23. The method of claim 22, wherein the second half dose is administered immediately or up to 2 hour after alcohol consumption.

24. The method of claim 17, wherein one dose of the oral composition is administered to the subject before or after alcohol consumption.
